(19) United States
(12) Patent Application Publication
(10) Pub. No.: US 2025/0231389 A1
(43) Pub. Date: Jul. 17, 2025

(54) HOLDER APPARATUS AND OBSERVATION APPARATUS

(71) Applicant: NIKON CORPORATION, Tokyo (JP)

(72) Inventors: Hitoshi Kawai, Saitama-shi (JP); Li-Jiun Chen, Hiroshima-shi (JP); Yuuya Takayama, Foster City, CA (US); Kumiko Matsui, Yokohama-shi (JP)

(73) Assignee: NIKON CORPORATION, Tokyo (JP)

(21) Appl. No.: 19/170,511

(22) Filed: Apr. 4, 2025

Related U.S. Application Data
(63) Continuation of application No. PCT/JP2023/042408, filed on Nov. 27, 2023.

(30) Foreign Application Priority Data
Dec. 6, 2022 (JP) 2022-194660

(51) Int. Cl. G02B 21/12; C12M 1/32; C12M 3/00; C12M 3/06

(52) U.S. Cl. CPC G02B 21/125; C12M 23/12; C12M 23/16; C12M 23/48

(57) ABSTRACT

A holder apparatus, which is placed on a stage of an observation apparatus including the stage, an observation optical system which is arranged below the stage and on which light from a biological sample is incident, and a control unit, includes: a holder body which holds a culture vessel in which the biological sample is arranged; and an illumination optical system which is provided in the holder body and illuminates the biological sample, and the illumination optical system is arranged in at least one of a side surface portion or an upper portion of the holder body when a direction in which the biological sample is observed is set as a vertical direction.

The contents of the following patent application(s) are incorporated herein by reference:
NO. 2022-194660 filed in JP on Dec. 6, 2022
NO. PCT/JP2023/042408 filed in WO on Nov. 27, 2023.

### BACKGROUND

### 1. Technical Field

The present invention relates to a holder apparatus and an observation apparatus.

### 2. Related Art

For a microfluidic device in which one or more channels are disposed, Patent Document 1 describes a microfluidic device observation apparatus and a microfluidic device observation method for observing a test object present inside the channels.

### PRIOR ART DOCUMENT

### Patent Document

Patent Document 1: International Publication No. 2020/021604

### General Disclosure

A first aspect of the present invention provides a holder apparatus including: a holder body which holds a culture vessel in which a biological sample is arranged; and an illumination optical system which is provided in the holder body and illuminates the biological sample.

The culture vessel may be a microfluidic device.

The culture vessel may be a well plate.

The holder body may hold a plurality of culture vessels including the culture vessel.

The illumination optical system may include an LED.

The illumination optical system may include a light guide part which guides light from outside.

The illumination optical system may be arranged in a side surface portion of the holder body when a direction in which the biological sample is observed is set as a vertical direction.

The illumination optical system may be arranged in an upper portion or a lower portion of the holder body when a direction in which the biological sample is observed is set as a vertical direction.

The illumination optical system may include at least one of a convex lens, a concave lens, or a light diffusion plate.

The illumination optical system may include a mask which blocks a part of light.

The illumination optical system may be detachable from the holder body.

The illumination optical system may be unitized.

The holder body may include a first body and a second body, the first body may have an upper portion, and the second body may have a side surface portion.

A second aspect of the present invention provides an observation apparatus including: a stage for placing a holder apparatus including a holder body which holds a vessel in which a biological sample is arranged, and an illumination optical system which is provided in the holder body and illuminates the biological sample; an observation optical system on which light from the biological sample is incident; and a control unit which controls the illumination optical system.

The control unit may be capable of controlling at least one of an intensity of illumination light, an illumination position of the illumination light with respect to the biological sample, or an illumination timing by controlling the illumination optical system.

The control unit may control the illumination optical system to perform dark field illumination or bright field illumination.

The summary clause does not necessarily describe all necessary features of the embodiments of the present invention. The present invention may also be a sub-combination of the features described above.

### BRIEF DESCRIPTION OF THE DRAWINGS

FIG. 1 is a top view illustrating an example of a schematic configuration of a microfluidic device 100 in a first embodiment.

FIG. 2 is a side view illustrating the example of the schematic configuration of the microfluidic device 100 in the first embodiment.

FIG. 3 is a top view illustrating an example of a schematic configuration of a holder apparatus 110 according to the first embodiment.

FIG. 4 is a side cross-sectional view illustrating the example of the schematic configuration of the holder apparatus 110 according to the first embodiment.

FIG. 5 is a perspective view illustrating the example of the schematic configuration of the holder apparatus 110 according to the first embodiment.

FIG. 6 is a side cross-sectional view illustrating an example of a schematic configuration of a holder apparatus 120 according to a second embodiment.

FIG. 7 is a side cross-sectional view illustrating an example of a schematic configuration of a holder apparatus 130 according to a third embodiment.

FIG. 8 is a side cross-sectional view illustrating an example of a schematic configuration of a holder apparatus 140 according to a fourth embodiment.

FIG. 9 is a side cross-sectional view illustrating an example of a schematic configuration of a holder apparatus 150 according to a fifth embodiment.

FIG. 10 is a side cross-sectional view illustrating an example of a schematic configuration of a holder apparatus 160 according to a sixth embodiment.

FIG. 11 is a side cross-sectional view illustrating an example of a schematic configuration of a holder apparatus 170 according to a seventh embodiment.

FIG. 12 is a side cross-sectional view illustrating an example of a schematic configuration of a holder apparatus 180 according to an eighth embodiment.

FIG. 13 is a side cross-sectional view illustrating an example of a schematic configuration of a holder apparatus 190 according to a ninth embodiment.

FIG. 14 is a side cross-sectional view illustrating an example of a schematic configuration of a holder apparatus 200 in a tenth embodiment.

FIG. 15 is a side cross-sectional view illustrating an example of a schematic configuration of a holder apparatus 210 in an eleventh embodiment.

FIG. 16 is a diagram illustrating an example of a schematic configuration of an observation apparatus 300 according to a twelfth embodiment.

FIG. 17 is a flowchart illustrating an example of an operation of the observation apparatus 300 in the twelfth embodiment.

FIG. 18 illustrates an example of a well plate 500.

FIG. 19 illustrates an example of a computer 2200.

### DESCRIPTION OF EXEMPLARY EMBODIMENTS

Hereinafter, the present invention will be described through embodiments of the invention. The following embodiments do not limit the invention according to the claims. Not all combinations of features described in the embodiments are essential for the solution of the invention.

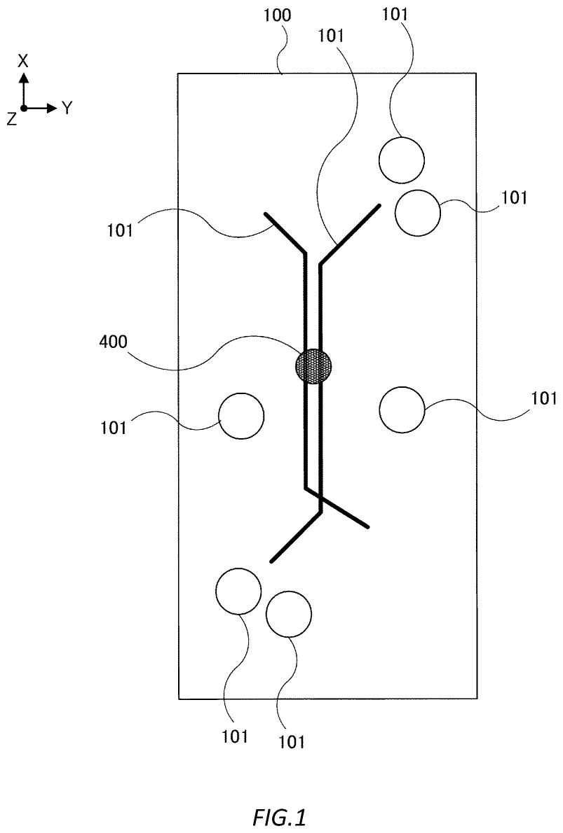

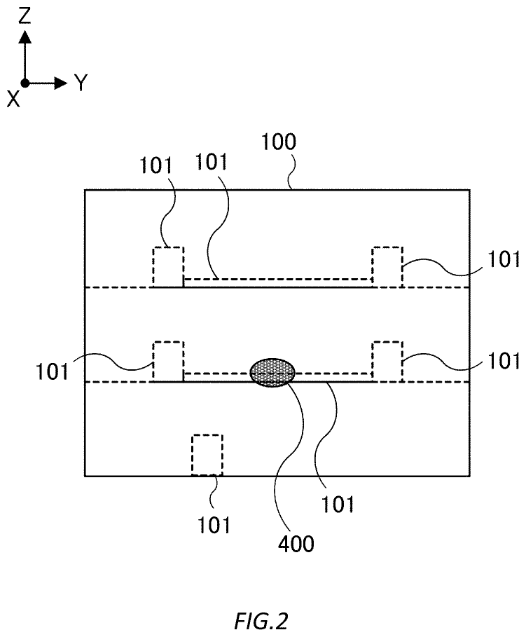

FIG. 1 is a top view illustrating an example of a schematic configuration of a microfluidic device 100 in a first embodiment. FIG. 2 is a side view illustrating the example of the schematic configuration of the microfluidic device 100 in the first embodiment. Hereinafter, an XYZ coordinate system is illustrated. As illustrated in FIGS. 1 and 2, the microfluidic device 100 has a substantially rectangular parallelepiped shape. Note that the present invention is also applicable to a microfluidic device having a different three-dimensional shape.

The microfluidic device 100 refers to a chip in which a sample corresponding to a biological fine substance such as DNA, protein, cell, cell mass (spheroids, organoids, or the like), or tissue is arranged on a small substrate, and genetic defects, protein distribution, reaction aspects, or the like are analyzed. The microfluidic device 100 in the present embodiment is also referred to as an organ on a chip, a biological function chip, a micro physiological system (MPS), a bio chip, a microfluidic chip, a micro chip, a cell culture chip, a microchannel chip, or the like. The microfluidic device 100 is a biological sample to be observed.

As an example, the microfluidic device 100 is used for culturing and analyzing cells, cell masses, and tissues. Furthermore, a chemical substance (drug) is added for use in evaluation or analysis of reactions with cultured cells or the like. The microfluidic device 100 may include both a device body in which organ cells have been cultured to exhibit biological functions and an “empty” device body in which organ cells have not yet been cultured.

The microfluidic device 100 can be manufactured, for example, by using stereolithography three-dimensional printing technology and a solution cast molding process. The microfluidic device 100 can be manufactured by another microfabrication technology such as micro electro mechanical systems (MEMS) in addition to the above technologies.

As illustrated in FIGS. 1 and 2, the microfluidic device 100 has, for example, a plurality of layers, and a plurality of structures 101 such as microchannels are arranged in each layer of the microfluidic device 100. For example, when a small intestine model is constructed, in the microchannels, an upper channel, mucus, a suction channel, small intestine epithelial cells, a porous membrane, endothelial cells, and a lower channel are layered in this order to construct the small intestine model. Note that the microfluidic device 100 is not limited to a case having a plurality of layers.

Each layer of the microfluidic device 100 is composed of a substrate as an example. The substrate may be composed of, for example, glass. The substrate may be composed of, for example, a resin material such as polymethyl methacrylate (PMMA), polycarbonate (PC), cycloolefin copolymer (COC), cycloolefin polymer (COP), polystyrene (PS), or silicon. The microfluidic device 100 may be hollow or solid. The microfluidic device 100 may have a cover which covers the entire microfluidic device 100. The microfluidic device 100 is desirably transparent to irradiation light and observation light.

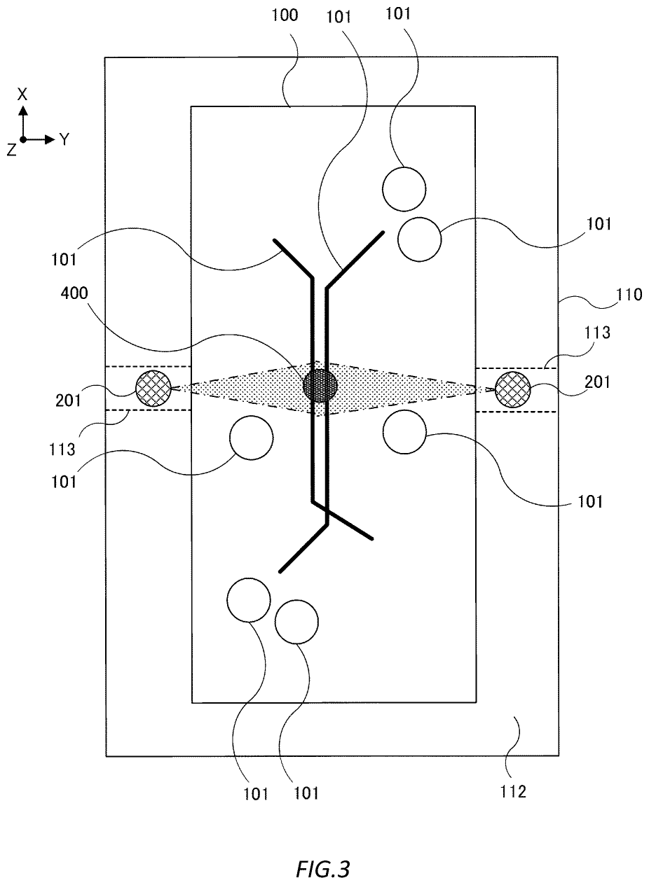

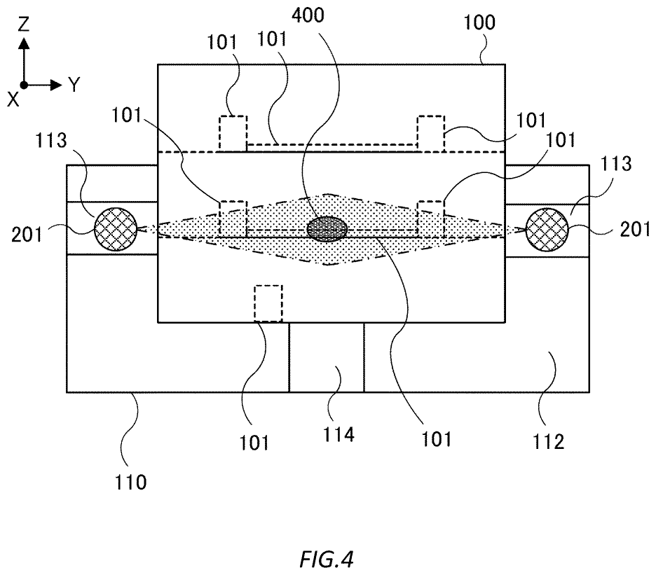

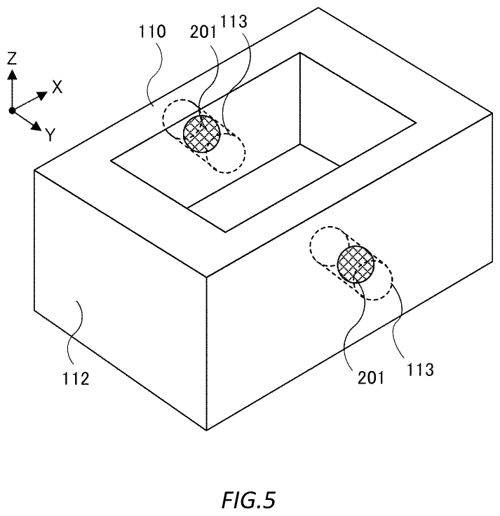

FIG. 3 is a top view illustrating an example of a schematic configuration of a holder apparatus 110 according to the first embodiment. FIG. 4 is a side cross-sectional view illustrating an example of the schematic configuration of the holder apparatus 110 according to the first embodiment. FIG. 5 is a perspective view illustrating the example of the schematic configuration of the holder apparatus 110 according to the first embodiment. The holder apparatus 110 in the first embodiment is an apparatus which holds the microfluidic device 100, which is a culture vessel, in the first embodiment. By holding the microfluidic device 100 with the holder apparatus 110, following effects are achieved: (1) since the microfluidic device 100 itself is small, holding the microfluidic device 100 with the holder apparatus 110 makes it easier to perform handling such as carrying, and (2) even if an outer shape of the microfluidic device 100 differs, standardizing an outer shape of the holder apparatus 110 allows the microfluidic device 100 to be handled without distinction of the outer shape of the microfluidic device 100 by attaching the microfluidic device 100 to the holder apparatus 110 corresponding to the device.

As illustrated in FIGS. 3 and 4, the holder apparatus 110 includes an illumination part 201. Hereinafter, a portion other than the illumination part 201 in the holder apparatus 110 may be referred to as a holder body 112. The holder body 112 may be made of, for example, resin or metal. A portion of the holder body 112 through which illumination light and observation light pass is opened. Alternatively, the entire holder body 112 may be transparent, or the portion through which the illumination light and the observation light pass may be transparent and other portions may be opaque. The illumination part 201 is an example of an illumination optical system. In the first embodiment, the illumination part 201 is formed as a light source which emits light by itself, for example, an LED. In another embodiment, the illumination part 201 may be formed as an optical waveguide including an optical fiber or the like. The illumination part 201 is arranged at the holder body 112, for example, inside the holder body 112. An opening 113 for inserting the illumination part 201 is formed substantially at a center of both side surfaces of the holder body 112 in an X direction, and the illumination part 201 is inserted into and fixed to the opening 113 of the holder body 112. The illumination light passes through the opening 113 for inserting the illumination part 201.

The illumination part 201 is arranged at an appropriate location according to a position of an observation region 400 in the microfluidic device 100. The observation region 400 of the microfluidic device 100 is a region which a user wants to observe in the microfluidic device 100, and is, for example, a region where cultured cells are arranged. In the observation region 400, for example, the structure 101 such as a microchannel, for example, a porous membrane or the like may be arranged in addition to the cultured cells. The observation region 400 may be a partial region of the porous membrane of the microfluidic device 100.

Arrangement of the observation region 400 in the microfluidic device 100 differs depending on the individual microfluidic device 100 and can be grasped in advance. Therefore, by considering a position of the holder apparatus 110 and the position of the observation region 400 in the microfluidic device 100 in a state where the holder apparatus 110 holds the microfluidic device 100, an appropriate position of the illumination part 201 in the holder apparatus 110 can be set in advance such that the illumination part 201 irradiates at least a part of the observation region 400.

In the first embodiment, since the observation region 400 of the microfluidic device 100 is near a center of the microfluidic device 100 in the X direction and a Y direction, the illumination parts 201 are arranged near centers of two side surfaces of the holder body 112 in the X direction so as to face each other in the Y direction. The illumination part 201 radiates the observation region 400 of the microfluidic device 100. For convenience, FIGS. 3 and 4 illustrate an example in which the illumination part 201 irradiates only a vicinity of the observation region 400. The illumination part 201 may be configured to be detachable from the holder body 112 (holder apparatus 110).

In the present embodiment, the illumination part 201 is arranged at a position not overlapping the observation region 400 of the microfluidic device 100 when viewed from an observation direction. The microfluidic device 100 in the first embodiment performs observation in a Z direction with an objective lens arranged on an upper surface or a lower surface. Hereinafter, a direction in which the observation region is observed is defined as a vertical direction. Therefore, the illumination part 201 is arranged on the side surface of the microfluidic device 100, which is a position not overlapping the observation region 400 when viewed from the Z direction. Note that the illumination part 201 may be arranged at a position overlapping the observation region 400 of the microfluidic device 100 when viewed from the observation direction. In the holder apparatus 110, an opening 114 for observation is provided to transmit the observation light.

A power source which supplies power to the illumination part 201 is connected to the illumination part 201 from outside. Note that the power source such as a button battery may be built in the holder apparatus 110 to supply power to the illumination part 201. In the present embodiment, the illumination parts 201 are arranged near the centers of the two side surfaces of the holder body 112 in the X direction so as to face each other in the Y direction, and thus are suitable for illumination of the observation region 400 near the center of the microfluidic device 100 in the X direction and the Y direction. For example, the irradiation light becomes excitation light in an ultraviolet region to an infrared region in a case of fluorescence observation, and becomes illumination light in a visible region to the infrared region in a case of bright field observation and dark field observation. The illumination part 201 is connected to an external control system, and the control system can control on/off, an illumination intensity, or the like of the illumination part 201. Note that the holder apparatus 110 may further include a mechanism for controlling a direction of illumination of the illumination part 201, for example, by rotating the illumination part 201 with respect to a predetermined axis.

The holder apparatus 110 according to the first embodiment includes the illumination part 201 which illuminates the observation region 400 in the microfluidic device 100 that is held. Accordingly, the observation region 400 can be appropriately illuminated and accurately observed.

According to the holder apparatus 110 in the first embodiment, the illumination part 201 is arranged at an appropriate location according to the position of the observation region 400 in the microfluidic device 100. Accordingly, the observation region 400 can be efficiently illuminated and accurately observed.

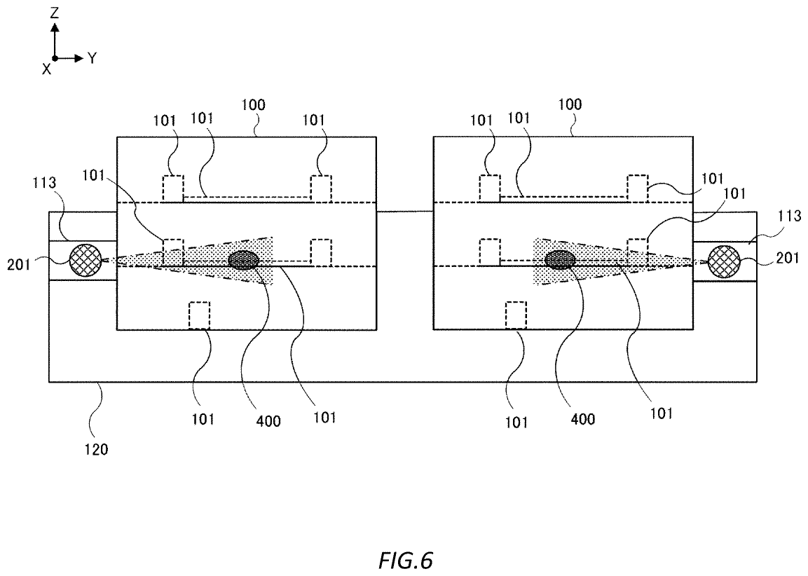

FIG. 6 is a side cross-sectional view illustrating an example of a schematic configuration of the holder apparatus 120 according to a second embodiment. Hereinafter, in description of the holder apparatus 120 of the second embodiment, identical components as those of the holder apparatus 110 of the first embodiment are denoted by same reference numerals, and description thereof will be omitted. Note that in FIG. 6 and subsequent drawings, illustration of some structures may be omitted.

As illustrated in FIG. 6, the holder apparatus 120 in the second embodiment holds two microfluidic devices 100 arranged side by side in the Y direction. The two illumination parts 201 are arranged on both side surfaces of the holder apparatus 120 in the Y direction. The illumination part 201 on a right side in FIG. 6 illuminates the observation region 400 in the microfluidic device 100 on the right side, and the illumination part 201 on a left side illuminates the observation region 400 in the microfluidic device 100 on the left side. The illumination part 201 in the second embodiment is formed as, for example, an LED. In another embodiment, the holder apparatus 120 may hold three or more microfluidic devices 100.

According to the holder apparatus 120 in the second embodiment, an effect similar to the effect achieved by the holder apparatus 110 in the first embodiment is achieved.

According to the holder apparatus 120 in the second embodiment, the holder apparatus 120 holds two microfluidic devices 100 arranged side by side in the Y direction. Therefore, the two microfluidic devices 100 can be efficiently illuminated and observed.

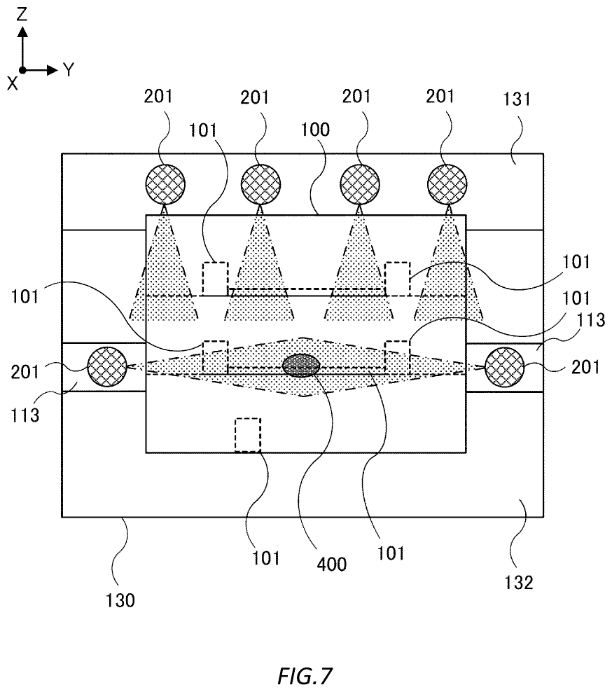

FIG. 7 is a side cross-sectional view illustrating an example of a schematic configuration of a holder apparatus 130 according to a third embodiment. Hereinafter, in description of the holder apparatus 130 of the third embodiment, identical components as those of the holder apparatus 110 of the first embodiment are denoted by same reference numerals, and description thereof will be omitted.

As illustrated in FIG. 7, the holder apparatus 130 according to the third embodiment includes a holder upper body 131 as a first body and a holder lower body 132 as a second body. The microfluidic device 100 is placed into the holder lower body 132 of the holder apparatus 130, and then is covered with the holder upper body 131 from above, whereby the holder apparatus 130 holds the microfluidic device 100.

As illustrated in FIG. 7, four illumination parts 201 are arranged in the holder upper body 131. The four illumination parts 201 arranged in the holder upper body 131 illuminate the observation region 400 of the microfluidic device 100 from above. Two illumination parts 201 are arranged in the openings 113 on both side surfaces of the holder lower body 132 in the X direction. The two illumination parts 201 arranged in the holder lower body 132 illuminate the observation region 400 of the microfluidic device 100 from sides.

According to the holder apparatus 130 in the third embodiment, an effect similar to the effect achieved by the holder apparatus 110 in the first embodiment is achieved.

According to the holder apparatus 130 in the third embodiment, the holder apparatus 130 includes the holder upper body 131 and the holder lower body 132, and the plurality of illumination parts 201 are arranged in the holder upper body 131 and the holder lower body 132 to simultaneously illuminate the observation region 400 of the microfluidic device 100 from above and from the sides. Therefore, the observation region 400 of the microfluidic device 100 can be illuminated from a plurality of directions and observed.

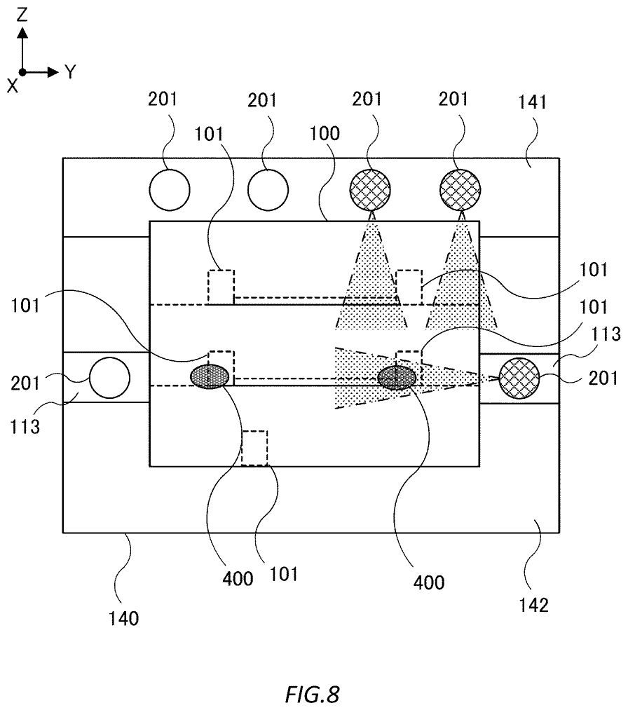

FIG. 8 is a side cross-sectional view illustrating an example of a schematic configuration of a holder apparatus 140 according to a fourth embodiment. Hereinafter, in description of the holder apparatus 140 of the fourth embodiment, identical components as those of the holder apparatus 110 of the first embodiment are denoted by same reference numerals, and description thereof will be omitted.

As illustrated in FIG. 8, the holder apparatus 140 according to the fourth embodiment includes a holder upper body 141 and a holder lower body 142. The microfluidic device 100 is placed into the holder lower body 142 of the holder apparatus 140, and then is closed by the holder upper body 141, whereby the holder apparatus 140 holds the microfluidic device 100. The microfluidic device 100 held by the holder apparatus 140 has two observation regions 400 in the Y direction (left and right) in a same layer.

As illustrated in FIG. 8, four illumination parts 201 are arranged in the holder upper body 141. The four illumination parts 201 arranged in the holder upper body 141 illuminate the observation region 400 of the microfluidic device 100 from above. Two illumination parts 201 are arranged in the openings 113 on both side surfaces of the holder lower body 142 in the X direction. The two illumination parts 201 arranged in the holder lower body 142 illuminate the observation region 400 of the microfluidic device 100 from the sides.

Each of the plurality of illumination parts 201 is configured to be switchable between on and off according to the position of the observation region 400. In the example illustrated in FIG. 8, the observation region 400 on a right side of the two observation regions 400 of the microfluidic device 100 is a region that the user wants to observe. In this case, the illumination part 201 close to the observation region 400 on the right side is controlled to be turned on. In FIG. 8, the illumination part 201 indicated by hatching is turned on, and the illumination part 201 indicated by white outline is turned off. Note that another on/off switching pattern is also applicable.

According to the holder apparatus 140 in the fourth embodiment, an effect similar to the effect achieved by the holder apparatus 110 in the first embodiment is achieved.

According to the holder apparatus 140 in the fourth embodiment, the holder apparatus 140 includes the holder upper body 141 and the holder lower body 142, and the plurality of illumination parts 201 are arranged in the holder upper body 141 and the holder lower body 142 to simultaneously illuminate the observation region 400 of the microfluidic device 100 from above and from the sides. Therefore, the microfluidic device 100 can be illuminated from a plurality of directions and observed.

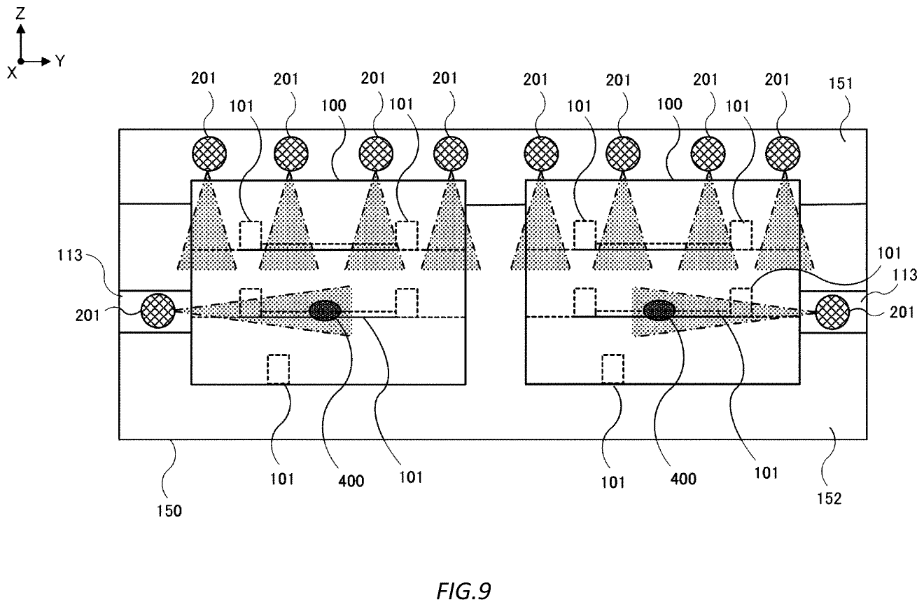

According to the holder apparatus 140 in the fourth embodiment, each of the plurality of illumination parts 201 of the holder apparatus 140 is configured to be switchable between on and off according to the position of the observation region 400. Therefore, observation can be performed with necessary and sufficient illumination according to the position of the observation region 400 of the microfluidic device 100. FIG. 9 is a side cross-sectional view illustrating an example of a schematic

configuration of a holder apparatus 150 according to a fifth embodiment. Hereinafter, in description of the holder apparatus 150 of the fifth embodiment, identical components as those of the holder apparatus 110 of the first embodiment are denoted by same reference numerals, and description thereof will be omitted.

As illustrated in FIG. 9, the holder apparatus 150 in the fifth embodiment holds two microfluidic devices 100 arranged side by side in the Y direction. The two illumination parts 201 are arranged in the openings 113 on both side surfaces of the holder apparatus 150 in the X direction. The illumination part 201 on a right side in FIG. 9 illuminates the observation region 400 of the microfluidic device 100 on the right side, and the illumination part 201 on a left side illuminates the observation region 400 of the microfluidic device 100 on the left side. The illumination part 201 in the fifth embodiment is formed as, for example, an LED. In another embodiment, the holder apparatus 120 may hold three or more microfluidic devices 100.

As illustrated in FIG. 9, the holder apparatus 150 according to the fifth embodiment includes a holder upper body 151 and a holder lower body 152. The microfluidic device 100 is placed into the holder lower body 152 of the holder apparatus 150, and then is closed by the holder upper body 151, whereby the holder apparatus 150 holds the microfluidic device 100.

As illustrated in FIG. 9, eight illumination parts 201 are arranged in the holder upper body 151. The eight illumination parts 201 arranged in the holder upper body 151 illuminate the observation region 400 of the microfluidic device 100 from above. Two illumination parts 201 are arranged in the openings 113 on both side surfaces of the holder lower body 152 in the X direction. The two illumination parts 201 arranged in the holder lower body 152 illuminate the observation region 400 of the microfluidic device 100 from the side. Note that each of the plurality of illumination parts 201 may be configured to be switchable between on and off according to the position of the observation region 400.

According to the holder apparatus 150 in the fifth embodiment, an effect similar to the effect achieved by the holder apparatus 110 in the first embodiment is achieved.

According to the holder apparatus 150 in the fifth embodiment, the holder apparatus 150 holds two microfluidic devices 100 arranged side by side in the Y direction. Therefore, the two microfluidic devices 100 can be efficiently illuminated and observed.

According to the holder apparatus 150 in the fifth embodiment, the holder apparatus 150 includes the holder upper body 151 and the holder lower body 152, and the plurality of illumination parts 201 are arranged in the holder upper body 151 and the holder lower body 152 to simultaneously illuminate the observation region 400 of the microfluidic device 100 from above and from the sides. Therefore, the observation region 400 of the microfluidic device 100 can be illuminated from a plurality of directions and observed.

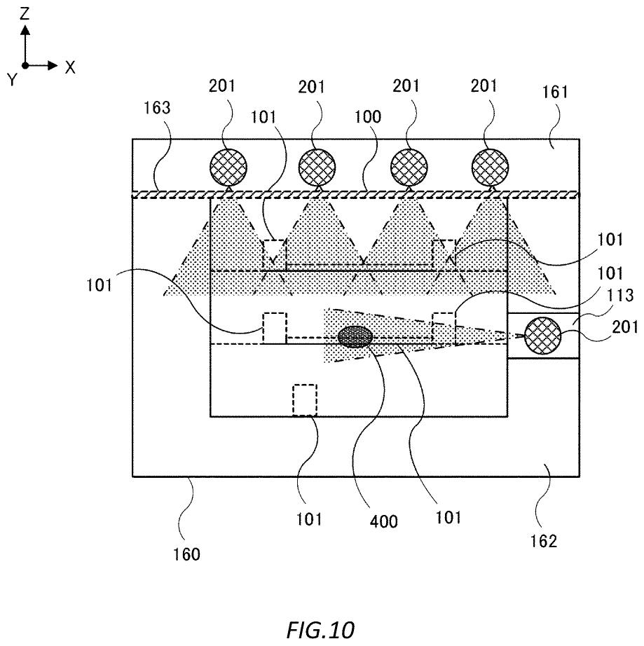

FIG. 10 is a side cross-sectional view illustrating an example of a schematic configuration of a holder apparatus 160 according to a sixth embodiment. Hereinafter, in description of the holder apparatus 160 of the sixth embodiment, identical components as those of the holder apparatus 110 of the first embodiment are denoted by same reference numerals, and description thereof will be omitted.

As illustrated in FIG. 10, the holder apparatus 160 according to the sixth embodiment includes a holder upper body 161 and a holder lower body 162. The microfluidic device 100 is placed into the holder lower body 162 of the holder apparatus 160, and then is closed by the holder upper body 161, whereby the holder apparatus 160 holds the microfluidic device 100. In addition, in the holder upper body 161, a light diffusion plate 163 is installed at a position which is a boundary with the holder lower body 162 and is in contact with the upper surface of the microfluidic device 100. The light diffusion plate 163 is an example of the illumination optical system.

As illustrated in FIG. 10, four illumination parts 201 are arranged in the holder upper body 161. The four illumination parts 201 arranged in the holder upper body 161 illuminate the observation region 400 of the microfluidic device 100 from above. Two illumination parts 201 are arranged in the openings 113 on both side surfaces of the holder lower body 162 in the X direction. The two illumination parts 201 arranged in the holder lower body 162 illuminate the observation region 400 of the microfluidic device 100 from the side.

The light diffusion plate 163 has a function of diffusing illumination light emitted from each of the four illumination parts 201 arranged in the holder upper body 161. The light diffusion plate 163 may be, for example, a lens diffusion plate (light shaping diffusers (LSD)) which diffuses and shapes light by a diffusion function of a lens array. As illustrated in FIG. 10, compared with the third embodiment, the illumination light from the four illumination parts 201 is diffused by the light diffusion plate 163.

According to the holder apparatus 160 in the sixth embodiment, an effect similar to the effect achieved by the holder apparatus 110 in the first embodiment is achieved.

According to the holder apparatus 160 in the sixth embodiment, the holder apparatus 160 includes the holder upper body 161 and the holder lower body 162, and the plurality of illumination parts 201 are arranged in the holder upper body 161 and the holder lower body 162 to simultaneously illuminate the observation region 400 of the microfluidic device 100 from above and from the sides. Therefore, the observation region 400 of the microfluidic device 100 can be illuminated from a plurality of directions and observed.

According to the holder apparatus 160 in the sixth embodiment, the light diffusion plate 163 is installed in the holder apparatus 160. Accordingly, the illumination light emitted from the illumination part 201 is diffused in a designed range, so that the light can be distributed in a necessary range. In addition, since the illumination light emitted from the illumination part 201 is diffused with high uniformity, illumination unevenness can be eliminated. Note that a uniformity of the illumination light can be further improved by arranging the light diffusion plate 163 close to the illumination part 201.

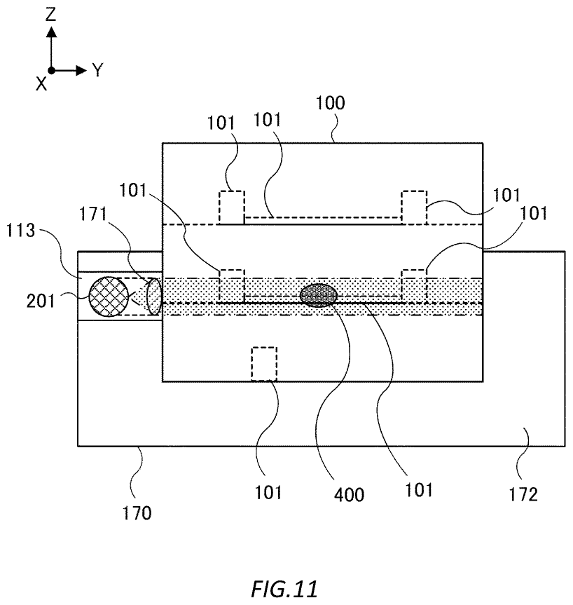

FIG. 11 is a side cross-sectional view illustrating an example of a schematic configuration of a holder apparatus 170 according to a seventh embodiment. Hereinafter, in description of the holder apparatus 170 of the seventh embodiment, identical components as those of the holder apparatus 110 of the first embodiment are denoted by same reference numerals, and description thereof will be omitted.

As illustrated in FIG. 11, the holder apparatus 170 according to the seventh embodiment includes the illumination part 201 and a convex lens 171 in each of the openings 113 on both side surfaces of a holder body 172 in the X direction. That is, the convex lens 171 is provided between the illumination part 201 and the microfluidic device 100. The convex lens 171 has a function of collimating light. The convex lens 171 may be, for example, a cylindrical lens. Therefore, the light emitted from the illumination part 201 is collimated into parallel light by passing through the convex lens 171. The convex lens 171 is an example of the illumination optical system. The illumination part 201 and the convex lens 171 may be unitized and configured to be detachable from the holder body 172 (holder apparatus 170).

According to the holder apparatus 170 in the seventh embodiment, an effect similar to the effect achieved by the holder apparatus 110 in the first embodiment is achieved.

According to the holder apparatus 170 in the seventh embodiment, the holder apparatus 170 includes the convex lens 171 between the illumination part 201 and the microfluidic device 100. Therefore, the light emitted from the illumination part 201 is collimated into parallel light, and only the vicinity of the observation region 400 can be efficiently illuminated.

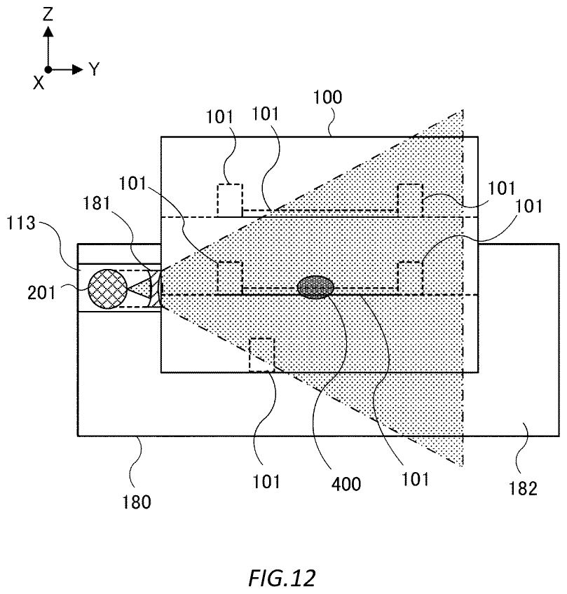

FIG. 12 is a side cross-sectional view illustrating an example of a schematic configuration of a holder apparatus 180 according to an eighth embodiment. Hereinafter, in description of the holder apparatus 180 of the eighth embodiment, identical components as those of the holder apparatus 110 of the first embodiment are denoted by same reference numerals, and description thereof will be omitted.

As illustrated in FIG. 12, the holder apparatus 180 according to the eighth embodiment includes the illumination part 201 and a concave lens 181 in each of the openings 113 on both side surfaces of a holder body 182 in the X direction. That is, the concave lens 181 is provided between the illumination part 201 and the microfluidic device 100. The concave lens 181 has a function of diffusing light. The concave lens 181 may be, for example, a cylindrical lens. Therefore, the light emitted from the illumination part 201 is diffused in a designed range by passing through the concave lens 181. The concave lens 181 is an example of the illumination optical system. The illumination part 201 and the concave lens 181 may be unitized and configured to be detachable from the holder body 182 (holder apparatus 180).

According to the holder apparatus 180 in the eighth embodiment, an effect similar to the effect achieved by the holder apparatus 110 in the first embodiment is achieved.

According to the holder apparatus 180 in the eighth embodiment, the holder apparatus 180 includes the concave lens 181 between the illumination part 201 and the microfluidic device 100. Therefore, the light emitted from the illumination part 201 is diffused in the designed range, and not only the vicinity of the observation region 400 but also other regions can be illuminated overall.

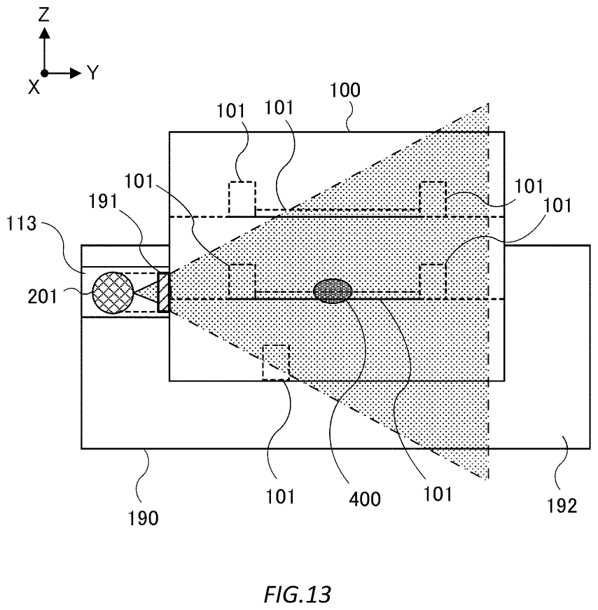

FIG. 13 is a side cross-sectional view illustrating an example of a schematic configuration of a holder apparatus 190 according to a ninth embodiment. Hereinafter, in description of the holder apparatus 190 of the ninth embodiment, identical components as those of the holder apparatus 110 of the first embodiment are denoted by same reference numerals, and description thereof will be omitted.

As illustrated in FIG. 13, the holder apparatus 190 according to the ninth embodiment includes the illumination part 201 and a light diffusion plate 191 in each of the openings 113 on both side surfaces of a holder body 192 in the X direction. That is, the light diffusion plate 191 is provided between the illumination part 201 and the microfluidic device 100. The light diffusion plate 191 has a function of diffusing light. The light diffusion plate 163 may be, for example, a lens diffusion plate which diffuses and shapes light by a diffusion function of a lens array. Therefore, the light emitted from the illumination part 201 is diffused by passing through the light diffusion plate 191. The light diffusion plate 191 is an example of the illumination optical system. The illumination part 201 and the light diffusion plate 191 may be unitized and configured to be detachable from the holder body 192 (holder apparatus 190).

According to the holder apparatus 190 in the ninth embodiment, an effect similar to the effect achieved by the holder apparatus 110 in the first embodiment is achieved.

According to the holder apparatus 190 in the ninth embodiment, the holder apparatus 190 includes the light diffusion plate 191 between the illumination part 201 and the microfluidic device 100. Therefore, the light emitted from the illumination part 201 is diffused in a designed range, and not only the vicinity of the observation region 400 but also other regions can be illuminated overall.

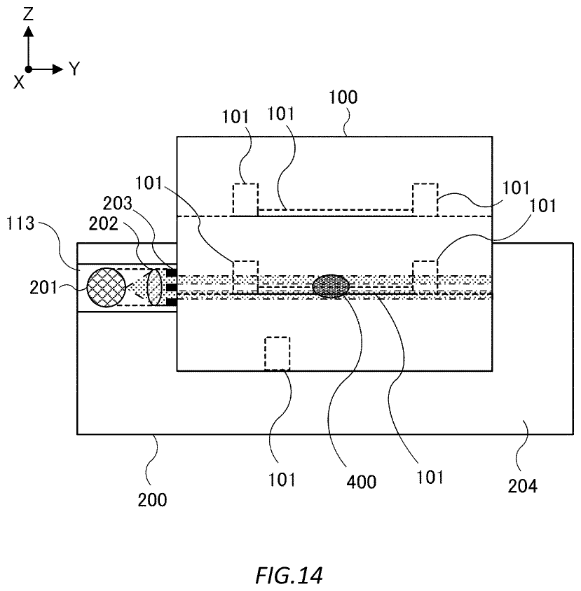

FIG. 14 is a side cross-sectional view illustrating an example of a schematic configuration of a holder apparatus 200 according to a tenth embodiment. Hereinafter, in description of the holder apparatus 200 of the tenth embodiment, identical components as those of the holder apparatus 110 of the first embodiment are denoted by same reference numerals, and description thereof will be omitted.

As illustrated in FIG. 14, the holder apparatus 200 according to the tenth embodiment includes the illumination part 201, a convex lens 202, and a mask 203 in each of the openings 113 on both side surfaces of a holder body 204 in the X direction. That is, the convex lens 202 and the mask 203 are provided between the illumination part 201 and the microfluidic device 100. The convex lens 202 has a function of collimating light. The convex lens 202 may be, for example, a cylindrical lens. Therefore, the light emitted from the illumination part 201 is collimated into parallel light by passing through the convex lens 202. The mask 203 has a function of blocking a part of the collimated light having passed through the convex lens 202. Therefore, the light emitted from the illumination part 201 is partially blocked and radiated to the observation region 400. The convex lens 202 and the mask 203 are examples of the illumination optical system. The illumination part 201, the convex lens 202, and the mask 203 may be unitized and configured to be detachable from the holder body 204 (holder apparatus 200).

According to the holder apparatus 200 in the tenth embodiment, an effect similar to the effect achieved by the holder apparatus 110 in the first embodiment is achieved.

According to the holder apparatus 200 in the tenth embodiment, the holder apparatus 200 includes the convex lens 202 and the mask 203 between the illumination part 201 and the microfluidic device 100. Therefore, the light emitted from the illumination part 201 is collimated into parallel light and partially blocked, and only a specific region in the observation region 400 can be illuminated.

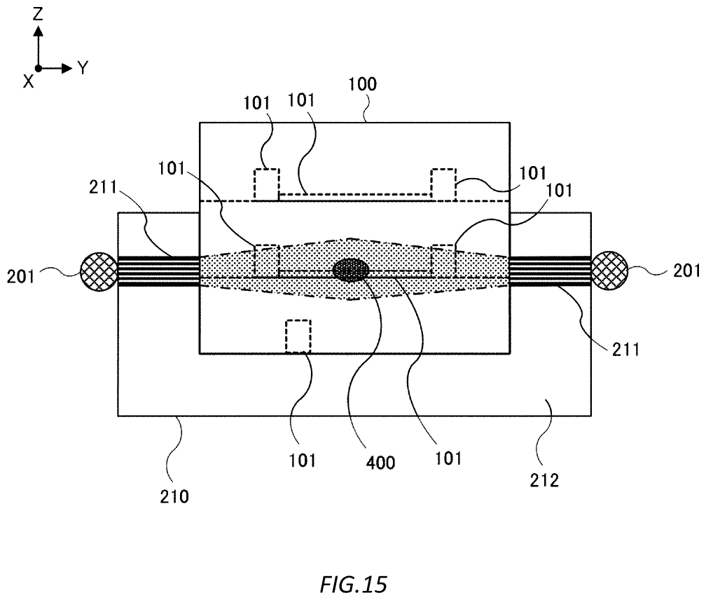

FIG. 15 is a side view illustrating an example of a schematic configuration of a holder apparatus 210 according to an eleventh embodiment. Hereinafter, in description of the holder apparatus 210 of the eleventh embodiment, identical components as those of the holder apparatus 110 of the first embodiment are denoted by same reference numerals, and description thereof will be omitted.

As illustrated in FIG. 15, the holder apparatus 210 according to the eleventh embodiment includes two illumination parts 201 at substantially centers of outer surfaces of both side surfaces of the holder body 212 in the X direction. Optical waveguides 211 as light guide parts, which guide illumination light from the two illumination parts 201 respectively, are provided inside substantially the centers of both side surfaces of the holder body 212 in the X direction (corresponding to positions where the two illumination parts 201 are arranged). That is, the optical waveguides 211 as the light guide parts are provided between the two illumination parts 201 and the observation region 400 of the microfluidic device 100. The optical waveguide 211 is an example of the illumination optical system. The optical waveguide 211 is a part for guiding the illumination light from the two illumination parts 201 to the observation region 400. The illumination part 201 may be configured to be detachable from the holder body 212 (holder apparatus 210).

The optical waveguide 211 is formed of a substance having a light refractive index higher than that of a material constituting the holder apparatus 210. Accordingly, without flowing out to an outside of the microfluidic device 100, the illumination light is guided to the observation region 400 of the microfluidic device 100 by the optical waveguide 211. The optical waveguide 211 is composed of, for example, quartz glass, silicon, high-purity polyimide-based resin, polyamide-based resin, polyether-based resin, or the like. The optical waveguide 211 may be selected in consideration of a transmittance, a refractive index, wavelength characteristics, a dispersibility, or the like of the illumination light.

According to the holder apparatus 210 in the eleventh embodiment, an effect similar to the effect achieved by the holder apparatus 110 in the first embodiment is achieved.

According to the holder apparatus 210 in the eleventh embodiment, the holder apparatus 210 includes the illumination part 201 and the optical waveguide 211. Therefore, the light emitted from the illumination part 201 is directed to the observation region 400 through the optical waveguide 211, and the observation region 400 can be appropriately illuminated.

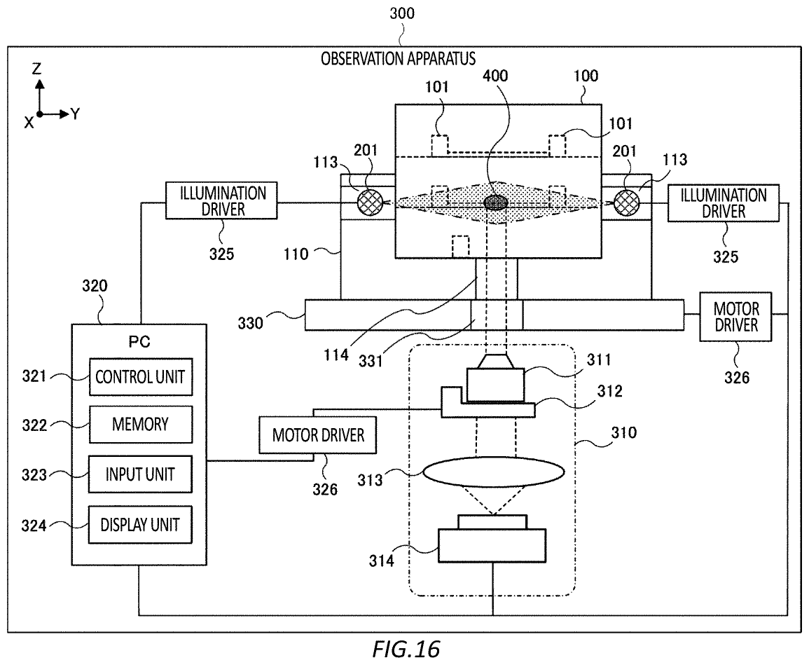

FIG. 16 illustrates an example of a schematic configuration of an observation apparatus 300 according to a twelfth embodiment. The observation apparatus 300 in the present embodiment may be an inverted microscope as an example. As illustrated in FIG. 16, the observation apparatus 300 includes an observation optical system 310, a personal computer (PC) 320, a first stage 330, an illumination driver 325, and a motor driver 326. As an example, the holder apparatus 110 according to the first embodiment is placed on the first stage 330 of the observation apparatus 300, and the holder apparatus 110 can be moved in the XY directions with respect to the observation optical system 310 by moving the first stage 330 in the XY directions. The holder apparatus 110 may be placed on the first stage 330 via an adapter (not illustrated). Note that the observation apparatus 300 is applicable to the observation of the microfluidic device in another embodiment. The holder apparatus 110 and the first stage 330 are provided with observation openings 114 and 331 for transmitting observation light.

The observation optical system 310 includes an objective lens 311, a second stage 312 on which the objective lens 311 is placed, an imaging lens 313, and a two-dimensional detector 314. The second stage 312 is movable in the Z direction (height direction), and can adjust a position of the objective lens 311 in the Z direction. The two-dimensional detector 314 detects observation light from the observation region 400. As an example, the two-dimensional detector 314 is an image sensor such as a charge coupled device (CCD) image sensor or a scientific complementary metal oxide semiconductor (sCMOS) image sensor.

The PC 320 includes a control unit 321 having a CPU and a memory 322, and the control unit 321 controls an operation of the observation apparatus 300 by reading and executing a control program stored in the memory 322. The PC 320 includes an input unit 323 which receives various instructions, settings, or the like from the user and transmits the instructions, settings, or the like to the control unit 321 of the PC 320, and a display unit 324 which receives a command from the control unit 321 and displays various dialogues and the like to the user.

As illustrated in FIG. 16, the PC 320 is connected with the first stage 330 and the second stage 312 of the observation apparatus 300 via the motor driver 326, and can control an operation of each stage by controlling the motor driver 326. In addition, as indicated by an alternate long and short dash line in FIG. 16, the PC 320 is connected with the two-dimensional detector 314, and an observed image is input thereto.

The PC 320 can control one or more of the illumination intensity, an illumination position, and an illumination timing of the illumination part 201. As illustrated in FIG. 16, the PC 320 is connected to the illumination part 201 in the holder apparatus 110 via the illumination driver 325, and can control the on/off and the illumination intensity of the illumination part 201 by controlling the illumination driver 325. In addition, the PC 320 can perform control such as blinking the illumination part 201 at a predetermined timing.

Note that the observation light detected by the two-dimensional detector 314 is light emitted from the illumination part 201 and transmitted/diffracted through the observation region 400 in the case of the bright field observation, and is light emitted from the illumination part 201 and reflected/diffracted/scattered in the observation region 400 in the case of the dark field observation. Whether to perform the bright field observation or the dark field observation can be set by optical characteristics of the observation optical system 310 such as a NA of the objective lens 311, a direction (angle) of the illumination light from the illumination part 201 to the observation region 400, or the like.

According to the observation apparatus 300 of the twelfth embodiment, an effect similar to as those of the holder apparatuses 110 to 210 of the first to eleventh embodiments can be achieved.

According to the observation apparatus 300 in the twelfth embodiment, the PC 320 can control one or more of the illumination intensity, the illumination position, and the illumination timing of the illumination part 201. Accordingly, appropriate illumination can be applied to the observation region 400 at a desired position.

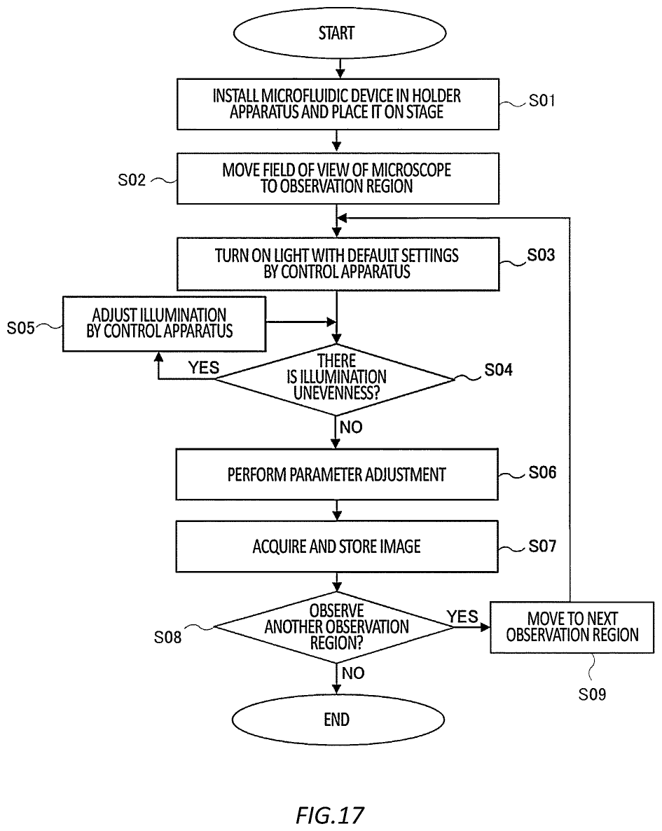

FIG. 17 is a flowchart illustrating an example of the operation of the observation apparatus 300 in the twelfth embodiment. In step S01, the microfluidic device 100 is installed in the holder apparatus 110 and placed on the first stage 330. Subsequently, in step S02, a field of view of a microscope as the observation apparatus 300 is moved to the observation region 400. Processing in step S02 is performed by the user manually adjusting the field of view of the microscope to the observation region 400. However, the processing in step S02 may be automatically performed by the observation apparatus 300.

Subsequently, in step S03, the PC 320 (control apparatus) controls the illumination part 201 to perform illumination with the illumination intensity, the illumination position, and the illumination timing set as default settings. Subsequently, in step S04, the PC 320 acquires an image of the observation region 400 and determines whether or not there is illumination unevenness in the observation region 400, based on the image. The illumination unevenness refers to a case where the illumination light of the illumination part 201 does not evenly reach the entire field of view, resulting in formation of a dark portion and a bright portion of the illumination. If there is illumination unevenness (YES in step S04), the process proceeds to step S05, and an amount of light or the like of the illumination part 201 is automatically adjusted by the PC 320. If there is no illumination unevenness (NO in step S04), the process proceeds to the next step S06, and the user adjusts various other parameters in the observation apparatus 300. Examples of the various parameters include a focal position of the objective lens, a magnification of the objective lens (switching of the objective lens), the illumination intensity, an exposure time/sensitivity of the two-dimensional detector, and the like.

Subsequently, in step S07, the PC 320 acquires and stores an image of the observation region 400. For example, the PC 320 may perform predetermined analysis and evaluation processing, such as counting of the number of cells, calculation of cell density distribution, and life/death determination of cells, on the stored image. Subsequently, in step S08, it is determined whether or not a next observation region 400 is present in the microfluidic device 100. If there is the next observation region 400 (step S08: YES), in the next step S09, the observation region moves to the next observation region, and the process returns to step S03 and repeats the processing from steps S03 to S07. If there is no next observation region (step S08: NO), the process ends.

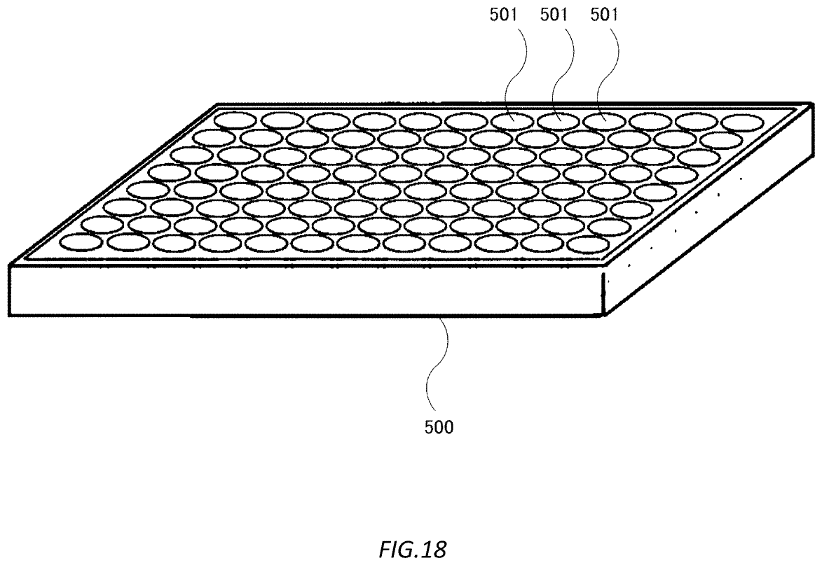

FIG. 18 illustrates an example of a well plate 500. In the above embodiments, the microfluidic device 100 has been described as an example of the culture vessel held by the holder apparatuses 110 to 210. However, the culture vessel held by the holder apparatuses 110 to 210 may be, for example, the well plate 500. The well plate 500 is an experimental and testing instrument composed of a flat plate with a large number of recesses 501 (holes or wells), and is used as a test tube or a petri dish for culturing cells or biological tissues. The well plate 500 is used for biochemical analysis, clinical examination, or the like.

In addition, various embodiments of the present invention may be described with reference to flowcharts and block diagrams, wherein the block may serve as (1) a stage in a process in which an operation is performed, or (2) a section of an apparatus having a role of performing an operation. Certain stages and sections may be implemented by a dedicated circuit, a programmable circuit supplied together with computer-readable instructions stored on computer-readable media, and/or processors supplied together with computer-readable instructions stored on computer-readable media. The dedicated circuit may include digital and/or analog hardware circuits, and may include integrated circuits (IC) and/or discrete circuits. The programmable circuit may include a reconfigurable hardware circuit including logical AND, logical OR, logical XOR, logical NAND, logical NOR, and other logical operations, a memory element or the like such as a flip-flop, a register, a field programmable gate array (FPGA) and a programmable logic array (PLA), or the like.

A computer-readable medium may include any tangible device that can store instructions to be executed by an appropriate device, and as a result, the computer-readable medium having instructions stored thereon includes a product including instructions that can be executed in order to create means for executing operations specified in the flowcharts or block diagrams. Examples of the computer-readable medium may include an electronic storage medium, a magnetic storage medium, an optical storage medium, an electromagnetic storage medium, a semiconductor storage medium, and the like. More specific examples of the computer readable medium may include a FLOPPY (registered trademark) disk, a diskette, a hard disk, a random access memory (RAM), a read-only memory (ROM), an erasable programmable read-only memory (EPROM or flash memory), an electrically erasable programmable read-only memory (EEPROM), a static random access memory (SRAM), a compact disc read-only memory (CD-ROM), a digital versatile disk (DVD), a BLU-RAY (registered trademark) disk, a memory stick, an integrated circuit card, or the like.

The computer-readable instruction may include: an assembler instruction, an instruction-set-architecture (ISA) instruction; a machine instruction; a machine dependent instruction; a microcode; a firmware instruction; state-setting data; or either a source code or an object code described in any combination of one or more programming languages, including an object oriented programming language such as SMALLTALK (registered trademark), JAVA (registered trademark), C++, or the like, and a conventional procedural programming language such as a “C” programming language or a similar programming language.

Computer-readable instructions may be provided to a processor of a general-purpose computer, special purpose computer, or other programmable data processing devices, or to programmable circuitry, locally or via a local area network (LAN), wide area network (WAN) such as the Internet, or the like, so that the computer-readable instructions are executed to create means for performing operations specified in the flowcharts or block diagrams. Examples of the processor include a computer processor, a processing unit, a microprocessor, a digital signal processor, a controller, a microcontroller, and the like.

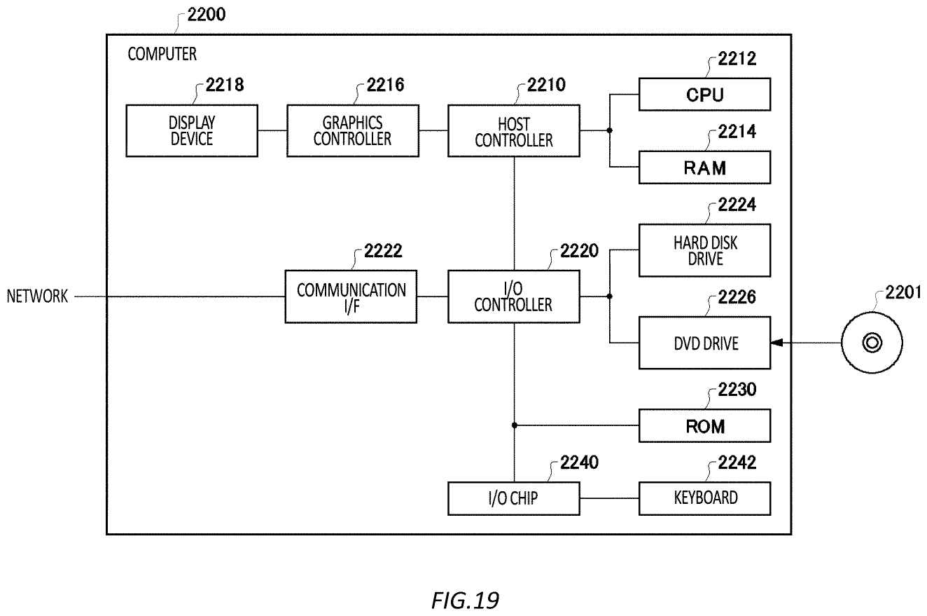

FIG. 19 illustrates an example of a computer 2200 in which a plurality of aspects of the present invention may be entirely or partially embodied. A program installed in the computer 2200 can cause the computer 2200 to function as an operation associated with the apparatuses according to the embodiments of the present invention or as one or more sections of the apparatuses, or can cause the operation or the one or more sections to be executed, and/or can cause the computer 2200 to execute a process according to the embodiments of the present invention or a stage of the process. Such programs may be executed by a CPU 2212 to cause the computer 2200 to perform specific operations associated with some or all of the blocks in the flowcharts and block diagrams described in the present specification.

The computer 2200 according to the present embodiment includes the CPU 2212, an RAM 2214, a graphics controller 2216, and a display device 2218, which are mutually connected by a host controller 2210. The computer 2200 also includes input/output units such as a communication interface 2222, a hard disk drive 2224, a DVD-ROM drive 2226, and an IC card drive, which are connected to the host controller 2210 via an input/output controller 2220. The computer also includes legacy input/output units such as an ROM 2230 and a keyboard 2242, which are connected to the input/output controller 2220 via an input/output chip 2240.

The CPU 2212 operates according to programs stored in the ROM 2230 and the RAM 2214, thereby controlling each unit. The graphics controller 2216 acquires image data generated by the CPU 2212 in a frame buffer or the like provided in the RAM 2214 or in itself, such that the image data is displayed on the display device 2218.

The communication interface 2222 communicates with other electronic devices via a network. The hard disk drive 2224 stores programs and data used by the CPU 2212 in the computer 2200. The DVD-ROM drive 2226 reads a program or data from a DVD-ROM 2201 and provides the program or data to the hard disk drive 2224 via the RAM 2214. The IC card drive reads the programs and the data from the IC card, and/or writes the programs and the data to the IC card.

The ROM 2230 stores therein boot programs and the like executed by the computer 2200 at the time of activation, and/or programs that depend on the hardware of the computer 2200. The input/output chip 2240 may also connect various input/output units to the input/output controller 2220 via a parallel port, a serial port, a keyboard port, a mouse port, or the like.

The program is provided by a computer readable medium such as the DVD-ROM 2201 or the IC card. The program is read from a computer readable medium, installed in the hard disk drive 2224, the RAM 2214, or the ROM 2230 which are also examples of the computer readable medium, and executed by the CPU 2212. The information processing described in these programs is read by the computer 2200 and provides cooperation between the programs and the above-described various types of hardware resources. The apparatus or method may be configured by implementing operations or processing of information according to use of the computer 2200.

For example, in a case where communication is performed between the computer 2200 and an external device, the CPU 2212 may execute a communication program loaded in the RAM 2214 and instruct the communication interface 2222 to perform communication processing based on a processing written in the communication program. Under the control of the CPU 2212, the communication interface 2222 reads transmission data stored in a transmission buffer processing region provided in a recording medium such as the RAM 2214, the hard disk drive 2224, the DVD-ROM 2201, or the IC card, transmits the read transmission data to the network, or writes reception data received from the network in a reception buffer processing region or the like provided on the recording medium.

In addition, the CPU 2212 may cause the RAM 2214 to read all or a necessary part of a file or database stored in an external recording medium such as the hard disk drive 2224, the DVD-ROM drive 2226 (DVD-ROM 2201), the IC card, or the like, and may execute various types of processing on data on the RAM 2214. Then, the CPU 2212 writes the processed data back in the external recording medium.

Various types of information such as various types of programs, data, tables, and databases may be stored in a recording medium and subjected to information processing. The CPU 2212 may execute, on the data read from the RAM 2214, various types of processing including various types of operations, information processing, conditional judgement, conditional branching, unconditional branching, information retrieval/replacement, or the like described throughout the present disclosure and specified by instruction sequences of the programs, and writes the results back to the RAM 2214. In addition, the CPU 2212 may retrieve information in a file, a database, or the like in the recording medium. For example, when a plurality of entries, each having an attribute value of a first attribute associated with an attribute value of a second attribute, is stored in the recording medium, the CPU 2212 may retrieve, out of the plurality of entries, an entry with the attribute value of the first attribute specified that meets a condition, read the attribute value of the second attribute stored in said entry, and thereby acquiring the attribute value of the second attribute associated with the first attribute meeting a predetermined condition.

The programs or software modules described above may be stored in a computer-readable medium on or near the computer 2200. In addition, a recording medium such as a hard disk or an RAM provided in a server system connected to a dedicated communication network or the Internet can be used as a computer-readable medium, thereby providing a program to the computer 2200 via the network.

While the present invention has been described by way of the embodiments, the technical scope of the present invention is not limited to the above-described embodiments. It is apparent to persons skilled in the art that various alterations or improvements can be made to the above described embodiments. It is also apparent from description of the claims that the embodiments to which such changes or improvements are made may be included in the technical scope of the present invention.

It should be noted that each process of the operations, procedures, steps, stages, and the like performed by the apparatus, system, program, and method shown in the claims, specification, or drawings can be executed in any order as long as the order is not indicated by “prior to”, “before”, or the like and as long as the output from a previous process is not used in a later process. Even if the operation flow is described using phrases such as “first” or “next” for the sake of convenience in the claims, specification, or drawings, it does not necessarily mean that the process must be performed in this order.

### EXPLANATION OF REFERENCES

100: microfluidic device; 101: structure; 110 to 210: holder apparatus; 112: holder body; 113: opening; 131: holder upper body; 132: holder lower body; 141: holder upper body; 142: holder lower body; 151: holder upper body; 152: holder lower body; 161: holder upper body; 162: holder lower body; 163: light diffusion plate; 171: convex lens; 172: holder body; 181: concave lens; 182: holder body; 191: light diffusion plate; 192: holder body; 201: illumination part; 202: convex lens; 203: mask; 204: holder body; 211: optical waveguide; 300: observation apparatus; 310: observation optical system; 311: objective lens; 312: second stage; 313: imaging lens; 314: two-dimensional detector; 320: PC; 321: control unit; 322: memory; 323: input unit; 324: display unit; 325: illumination driver; 326: motor driver; 330: first stage; 400: observation region; 500: well plate; 2200: computer; 2201: DVD-ROM; 2210: host controller; 2212: CPU; 2214: RAM; 2216: graphics controller; 2218: display device; 2220: input/output controller; 2222: communication interface; 2224: hard disk drive; 2226: DVD-ROM drive; 2230: ROM; 2240: input/output chip; and 2242: keyboard.

What is claimed is:

1. A holder apparatus which is placed on a stage of an observation apparatus including the stage, an observation optical system which is arranged below the stage and on which light from a biological sample is incident, and a control unit, the holder apparatus comprising:
a holder body which holds a culture vessel in which the biological sample is arranged; and
an illumination optical system which is provided in the holder body and illuminates the biological sample,
wherein
the illumination optical system is arranged in at least one of a side surface portion or an upper portion of the holder body when a direction in which the biological sample is observed is set as a vertical direction.

2. The holder apparatus according to claim 1, wherein the culture vessel is a microfluidic device.

3. The holder apparatus according to claim 1, wherein the culture vessel is a well plate.

4. The holder apparatus according to claim 1, wherein the holder body holds a plurality of culture vessels including the culture vessel.

5. The holder apparatus according to claim 1, wherein the illumination optical system includes an LED.

6. The holder apparatus according to claim 1, wherein the illumination optical system includes a light guide part which guides light from outside.

7. The holder apparatus according to claim 1, wherein the illumination optical system includes at least one of a convex lens, a concave lens, or a light diffusion plate.

8. The holder apparatus according to claim 1, wherein the illumination optical system includes a mask which blocks a part of light.

9. The holder apparatus according to claim 1, wherein the illumination optical system is detachable from the holder body.

10. The holder apparatus according to claim 1, wherein the illumination optical system is unitized.

11. The holder apparatus according to claim 1, wherein the holder body includes a first body and a second body, the first body has an upper portion, and the second body has a side surface portion.

12. The holder apparatus according to claim 1, wherein the illumination optical system controls at least one of an intensity of illumination light, an illumination position of the illumination light with respect to the biological sample, or an illumination timing, based on a control signal from the control unit.

13. The holder apparatus according to claim 1, wherein the control unit controls the illumination optical system to perform dark field illumination or bright field illumination.

14. The holder apparatus according to claim 1, wherein the holder apparatus includes a plurality of illumination optical systems including the illumination optical system, and on/off control of each of the plurality of illumination optical systems is performed according to a position of the biological sample, based on a control signal from the control unit.
